(19) United States
(12) Plant Patent Application Publication
(10) Pub. No.: US 2016/0316607 P1
(43) Pub. Date: Oct. 27, 2016

(54) Dahlia plant named 'KLEDH14045'

(50) ;

(71) Applicant: Klemm+Sohn GmbH & Co. KG, Stuttgart (DE)

(72) Inventors: Nils Klemm, Stuttgart (DE); Silke Wagener, Leinfelden-Echterdingen (DE)

(73) Assignee: Klemm+Sohn GmbH & Co. KG, Stuttgart (DE)

(21) Appl. No.: 14/545,319

(22) Filed: Apr. 22, 2015

(51) Int. Cl. A01H 5/00

(52) U.S. Cl. PLT/321

(57) ABSTRACT

A new Dahlia plant particularly distinguished by having yellow ray florets and a medium plant height, is disclosed.

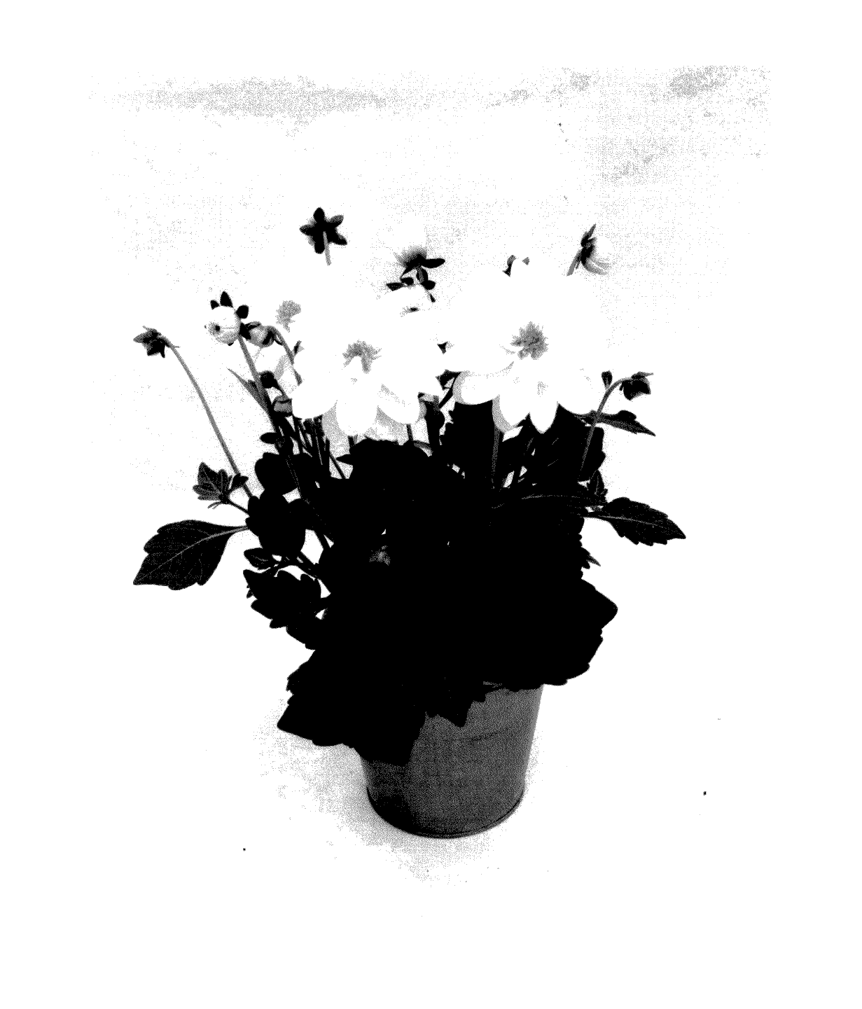

### GENUS AND SPECIES

Dahlia sp.

### VARIETY DENOMINATION

‘KLEDH 14045’

### BACKGROUND OF THE NEW PLANT

The present invention comprises a new and distinct variety of Dahlia, botanically known as Dahlia sp. and hereinafter referred to by the variety name ‘KLEDH14045’. ‘KLEDH 14045’ is a selection that resulted from an open pollination of the proprietary female dahlia variety ‘DH-2010-0329’ (unpatented) and an unknown male parent in August and September of 2010. Seed were collected from the open pollination and planted, with a single plant selected in Stuttgart, Germany in May of 2011 and subsequently named ‘KLEDH14045’. Initial terminal cuttings of ‘KLEDH14045’ were taken in May of 2011 in Stuttgart, Germany. ‘KLEDH 14045’ was found to reproduce true to type in successive generations of asexual propagation via terminal cuttings in Stuttgart, Germany.

### SUMMARY

The following are the most outstanding and distinguishing characteristics of this new variety when grown under normal horticultural practices in Stuttgart, Germany.

1. Yellow ray florets; and

2. A medium plant height.

### DESCRIPTION OF THE PHOTOGRAPHS

This new Dahlia plant is illustrated by the accompanying photograph which shows the overall plant habit of the plant grown in a pot. The plant is about 3 months-old grown in Stuttgart, Germany in April 2014. The colors shown are as true as can be reasonably obtained by conventional photographic procedures.

### DESCRIPTION OF THE NEW VARIETY

The following detailed descriptions set forth the distinctive characteristics of ‘KLEDH14045’. The data which define these characteristics were collected from asexual reproductions carried out in Stuttgart, Germany. Data was collected on two month-old plants in April 2015, grown in a controlled greenhouse environment approximate to those generally used in commercial production in Germany, natural light, unpinched, with growth regulators DAZIDE® and CYCOCEL®. Color references are to The R.H.S. Colour Chart of The Royal Horticultural Society of London (R.H.S.), 5^{th} edition (2007).
Classification:
   Family.—Asteraceae.
   Species.—Dahlia sp.
   Common name.—Dahlia.
   Variety.—‘KLEDH14045’.
Parentage:
   Female.—The proprietary female dahlia ‘DH-2010-0329’ (unpatented).
   Male.—Unknown.
Propagation:
   Type.—Asexual, terminal cuttings.
   Time to produce a rooted young plant.—3 to 4 weeks.
   Root description.—Medium thickness, light tan color.
   Rooting habit.—Freely branching, moderately dense.
Plant description:
   Plant and growth habit.—Compact, semi-upright, freely basal branching, inflorescences held above the foliar plane.
   Plant height.—About 30.0 cm.
   Plant diameter.—About 28.0 cm.
Lateral branches:
   Length.—16.0 cm.
   Diameter.—About 0.4 cm.
   Internode length.—Between 5.0 cm and 9.0 cm.
   Aspect.—Outwardly spreading.
   Strength.—Strong.
   Texture.—Smooth.
   Color.—RHS 144C.
Foliage description:
   Arrangement.—Leaves opposite, pinnate leaf type.
   Length.—About 13.0 cm.
   Width.—About 10.0 cm.
   Shape.—Oval.
   Apex.—Acute.
   Base.—Acute.
   Margin.—Moderate serrate.
   Venation pattern.—Pinnate.
   Venation color.—RHS 145B.
   Texture.—Upper and lower surfaces: Pilose leaves.
   Color.—Immature: Upper surface: RHS 143B Lower surface: RHS 139D Mature: Upper surface: RHS 143A Lower surface: RHS 139C.
Petioles:
   Length.—2.0 cm to 3.0 cm.
   Diameter.—About 0.3 cm.
   Texture.—Upper and lower surfaces: Smooth.
   Color.—Upper and lower surfaces: RHS 144C.
Inflorescence description:
   Appearance and arrangement.—Daisy-eyed double inflorescences above the foliar plane.
   Fragrance.—None detected.
   Time to flower.—Winter: About 3 months after planting Summer: About 2 months after planting.
   Post-production longevity.—Plants flower continuously during summer and autumn in Germany.
Inflorescence bud:
   Height.—Between 1.0 cm and 2.0 cm.
   Diameter.—Between 1.5 cm and 2.5 cm.
   Shape.—Oblate.
   Color.—RHS N144C.
Inflorescence size:
   Diameter.—About 9.0 cm.
   Depth (height).—About 3.0 cm.
   Disc diameter.—About 1.0 cm.
   Receptacle height.—About 1.0 cm.
   Receptacle diameter.—About 3.5 cm.
   Receptacle color.—Close to RHS 144C.
Ray florets:
   Quantity per inflorescence.—About 40 arranged in about 3 rows.
   Length.—Between 3.5 cm and 4.0 cm.
   Width.—Between 2.0 cm and 2.5 cm.
   Shape.—Elliptic.
   Apex.—Acute to obtuse.
   Base.—Rounded.
   Margin.—Entire.
   Aspect.—Semi-upright.
   Texture.—Upper and lower surfaces: Smooth.
   Color.—Immature: Upper and lower surfaces: RHS 2A Mature: Upper surface: RHS 1A and RHS 2A Lower surface: RHS 1B.
Disc florets:
   Quantity per inflorescence.—About 30.
   Length.—About 1 cm.
   Diameter.—0.2 cm.
   Shape.—Tubular.
   Texture.—Inner and outer surface: Smooth.
   Color.—Immature: Inner and outer surfaces: RHS 3B Mature: Inner and outer surfaces: RHS 13A.
Phyllaries:
   Quantity per inflorescence.—6.
   Length.—1.5 cm to 2.0 cm.
   Width.—About 0.8 cm.
   Shape.—Oval.
   Apex.—Acute.
   Base.—Acute.
   Margin.—Entire.
   Texture.—Upper and lower surfaces: Smooth and shiny.
   Color.—Upper surface: RHS 141C Lower surface: RHS 141B.
Peduncles:
   Length, terminal peduncle.—About 20.0 cm.
   Length, axillary peduncle.—About 10.0 cm.
   Diameter.—0.3 cm.
   Aspect.—Semi-upright.
   Strength.—Strong.
   Texture.—Smooth.
   Color.—RHS 144C.
Reproductive organs:
   Androecium.—Present on disc florets only Quantity per floret: 4 Filament length: 0.2 cm Filament color: RHS 4D Anther shape: Long, lanceolate Anther length: 0.5 cm Anther color: RHS 15D Pollen amount: Abundant Pollen color: RHS 17A.
   Gynoecium.—Quantity per floret: 1 Pistil length: 0.9 cm Style length: 0.4 cm Style color: RHS 5C Stigma shape: Lanceolate pinnate Stigma color: RHS 13A Ovary color: RHS 150D.
Fruit and seed set: None observed
Disease and insect/pest resistance: No particular disease or insect resistance observed

### COMPARISON WITH FEMALE PARENTAL LINE AND KNOWN VARIETY

‘KLEDH14045’ is a distinct variety of dahlia. ‘KLEDH14045’ is distinguished from its female parent as described in Table 1:

TABLE 1
Comparison with Female Parent

| | Variety | Female parent |
|---|---|---|
| Trait | ‘KLEDH14045’ | ‘DH-2010-0329’ |
| Plant height | Yellow | Purple and yellow |

‘KLEDH 14045’ is most similar to the commercial Dahlia plant named ‘Baldelemz’ (U.S. Plant Pat. No. 16,448), commercially known as Delicious Lemon Zest. Differences between the two varieties are described in Table 2:

TABLE 2
Comparison with Known Variety

| | Variety | Commercial line |
|---|---|---|
| Trait | ‘KLEDH14045’ | ‘Baldelemz’ |
| Plant height | Medium | Short to medium |

We claim:

1. A new and distinct variety of Dahlia plant named ‘KLEDH14045’ as illustrated and described herein.
